(19) United States
(12) Patent Application Publication
(10) Pub. No.: US 2009/0170132 A1
(43) Pub. Date: Jul. 2, 2009

(54) Methods for detecting colon carcinoma

(76) Inventor: Paul H. Pevsner, New York, NY (US)

Correspondence Address: KLAUBER & JACKSON, 411 HACKENSACK AVENUE, HACKENSACK, NJ, 07601, US

(21) Appl. No.: 12/316,374

(22) Filed: Dec. 11, 2008

Related U.S. Application Data
(60) Provisional application No. 61/007,225, filed on Dec. 11, 2007.

(51) Int. Cl. G01N 33/574; G01N 33/68; G01N 33/72; C12Q 1/32; G01N 33/566

(52) U.S. Cl. 435/7.23; 436/86; 436/66; 435/26; 436/501

(57) ABSTRACT

The present invention provides methods for diagnosing carcinoma, providing a prognosis for a carcinoma or assessing the likelihood that a tissue may become cancerous by identifying the presence or absence of or determining the amount of one or more carcinoma associated markers. Further, the present invention provides methods for determining whether a tissue should be surgically resected and for determining the territorial extent of resection. The carcinoma markers may be those provided in FIG. 1. The present invention also provides a diagnostic kit for diagnosing carcinoma, providing a prognosis for a carcinoma or assessing the likelihood that a tissue may become cancerous by identifying the presence of or determining the amount of one or more carcinoma associated markers. The methods and kits are especially useful regarding colon, rectal or colorectal carcinoma.

### CROSS REFERENCE TO RELATED APPLICATIONS

The present application claims the benefit of U.S. provisional patent application 60/007,225, filed on Dec. 11, 2007, the disclosure of which is herein incorporated by reference.

### FIELD OF THE INVENTION

This invention relates to methods of diagnosing carcinoma, providing a prognosis for a carcinoma or assessing the likelihood that a tissue may become cancerous.

### BACKGROUND OF THE INVENTION

Colorectal cancer (CRC) is the second-leading cause of death from cancer in the USA, with more than 155,000 predicted new cases per year. (Vogelaar et al., Cancer 2006; 107:1624-1633) The projected number of colorectal cancer deaths for 2008 is 53,000. The standard diagnostic paradigm is based on histopathology of either biopsy or surgical specimens.

In an extensive study, Song et al. demonstrated that fecal DNA testing every 5 years appeared both diagnostically effective and cost-effective compared with no screening, but inferior to other strategies such as fecal occult blood testing, FOBT, and colonoscopy. Fecal DNA testing could decrease the national burden if it could improve adherence with screening, particularly where the capacity to perform screening colonoscopy is limited. (Song, et al. Gastroenterology 2004; 126:1270-9.) In a follow-up study, the potential national impact of widespread screening on clinical outcomes (with screening uptake of 75%) were as follows: the incidence of colorectal cancer (CRC) could decrease by 17%-54% to as few as 66,000 new cases per year, and the deaths could decrease by 28%-60% to as few as 23,000 per year, depending on the strategy. Screening colonoscopy every 10 years would require 8.1 million colonoscopies per year including surveillance, with other strategies requiring 17%-58% as many colonoscopies. The current national endoscopic capacity, as recently estimated, may be adequate to support widespread use of screening colonoscopy in the steady state. The impact of emerging tests on colonoscopy demand will depend on the extent to which they replace screening colonoscopy or increase screening uptake in the population. With improved screening uptake, total colonoscopy demand would increase, even assuming substantial use of virtual colonoscopy. The conclusion was that despite savings in care, widespread screening is unlikely to be cost saving and may increase national expenditures by $0.8-$2.8 billion per year with conventional tests. (Ladabaum et al., Gastroenterology 2005; 129:1151-62) Even with surveillance, the prevalence and mortality of CRC cancer increases every year.

In 1953, Slaughter et al. introduced the concept of field cancerization in head and neck tumors. They described this as an area of epithelium that has been preconditioned by an as yet unknown carcinogenic agent. (Slaughter et al., Cancer 1953; 6:963-8) Braakhuis et al. suggested a genetic explanation for “field cancerization”, the carcinogenesis model in which the development of a field with genetically altered cells plays a central role. In the initial phase, a stem cell acquires genetic alterations and forms a “patch,” or clonal unit of altered daughter cells. These patches can be recognized based on mutations in tumor suppressor protein, TP53, and have been reported for head and neck, lung, skin, and breast cancer. The conversion of a patch into an expanding field is the next logical and critical step in epithelial carcinogenesis. Additional genetic alterations are required for this step, and by virtue of its growth advantage, a proliferating field gradually displaces the normal mucosa. In the mucosa of the head and neck, as well as the esophagus, such fields have been detected with dimensions of >7 cm in diameter. They are usually not detected by routine diagnostic techniques. Ultimately, clonal divergence leads to the development of one or more tumors within a contiguous field of preneoplastic cells. An important clinical implication is that fields often remain after surgery of the primary tumor and may lead to new cancers. These are designated presently by clinicians as “a second primary tumor” or “local recurrence,” depending on the exact site and time interval. The development of an expanding preneoplastic field appears to be a critical step in epithelial carcinogenesis with important clinical consequences. The diagnosis and treatment of epithelial cancers should not only be focused on the tumor but also on the field from which it developed. (Braakhuis et al., Cancer Res 2003; 63:1727-30.) Buckley et al. described HCG in sixty cases of adenocarcinoma of the colon and none in control normal tissue. (Buckley et al., J Clin Pathol 1979; 32:368-72.) Subsequently, hCG was described by immunohistochemistry in infiltrating rectosigmoid carcinoma and in some of the histologically normal satellite tissue adjacent to carcinomas. (Fukayama et al., Am J Pathol 1987; 127:83-9; Yamaguchi et al., Br J Cancer 1989; 60:382-4.)

Fearon et al. presented a genetic model for colorectal cancer (CRC) tumor genesis. He proposed that CRC tumors appear to arise as a result of the mutational activation of oncogenes coupled with the mutational inactivation of tumor suppressor genes (which predominate). Second, mutations in at least four to five genes are required for the formation of a malignant tumor. Fewer changes suffice for benign tumor genesis. Third, although the genetic alterations often occur according to a preferred sequence, the total accumulation of changes, rather than their order with respect to one another, is responsible for determining the tumor's biologic properties. Fourth, in some cases, mutant tumor suppressor genes appear to exert a phenotypic effect even when present in the heterozygous state; thus, some tumor suppressor genes may not be “recessive” at the cellular level. The general features of this model may be applicable to other common epithelial neoplasms. They further proposed that an epigenetic change, like hypomethylation, could contribute to instability in the tumor cell genome and alter the rate at which genetic alterations, such as allelic losses, occur. He observed that the carcinomatous regions were derived from (and not simply adjacent to) the adenomatous regions. This was proven in several cases by the finding that the identical ras gene mutation was present in both regions. In all cases, however, the carcinomatous regions contained at least one alteration not found in the adenomatous region. (Fearon et al., Cell 1990; 61:759-67) Scalmati et al. described changes in the normal mucosa of patients with CRC carcinoma. The normal-appearing rectal mucosa of the affected patients showed a pattern of cell proliferation that clearly differs from that of normal people. The proliferative compartment of normal CRC mucosa of individuals at low risk for CRC cancer, and of normal rodents, is located in the lower part of the colonic crypts. The total labeling index (i.e., labeled cells versus total cells) of low-risk controls and familial polyposis patients does not differ significantly. However, the patients show a shift of the proliferative compartment toward the top of the crypts. Thus, the main proliferative alteration noted in the colonic mucosa of these patients is not an increase in overall cell proliferation labeling index, but a lumenward displacement of the zone of active cell proliferation in the crypts. (Scalmati et al., Environ Health Perspect 1993; 99:169-73)

Aberrant crypt foci (ACF*) are putative preneoplastic lesions of colonic cancer. A diet containing 0.2% cholic acid (CHA), a reported colonic tumor promoter, has two prominent effects on the growth of ACF: (a) a reduction in the number of ACF present in the colon due to either elimination or remodeling and (b) enhanced growth of remaining ACF to colonic cancer. (Magnuson et al., Carcinogenesis 1994; 15:1459-62).

Increased bcl-2 expression was present in hyperplastic colonic polyps and in the majority of dysplastic lesions, from the earliest precursors through large adenomas, high grade flat dysplasia, and adenocarcinoma. Furthermore, bcl-2 expression was frequently abnormal in nondysplastic epithelium surrounding dysplastic lesions, suggesting that altered expression occurred before the development of morphological dysplasia. Specifically, directly contiguous morphologically nondysplastic epithelium often showed abnormal bcl-2 expression throughout the full length of the crypt-villus axis. This expression pattern gradually diminished to involve only the crypt base (the normal pattern of expression), proceeding away from malignant or dysplastic lesions. Abnormal bcl-2 immunoreactivity in 1), the earliest precursor dysplastic lesions and its persistence throughout neoplastic progression and 2), contiguous morphologically unaltered nondysplastic epithelium suggests that bcl-2 alterations occur early during the morphological and molecular sequence of events leading to gastrointestinal epithelial neoplasia. (Bronner et al., Am J Pathol 1995; 146:20-6)

Garewal et al. proposed an assay for quantitation of bile acid-induced reduction in apoptotic ability of CRC mucosa which he implied increased cancer risk. By applying a quantitative bile acid-induced apoptosis assay to CRC mucosal biopsies, the percentage of apoptosis was found to be significantly reduced in CRC carcinoma patients. Furthermore, he proposed that cells with mutations and resistance to apoptosis in “normal” appearing mucosa represented a field defect that could potentially become carcinoma. (Garewal et al., Cancer Res 1996; 56:1480-3)

A novel antiapoptosis gene, designated “survivin” is an inhibitor of apoptosis protein (IAP), a group of proteins known to inhibit caspases, the proteolytic components of the apoptotic pathway implicated in the control of cell cycle progression. Survivin localizes to the inter-mitochondrial membrane space in tumor cells. This localization accelerates tumor genesis in vivo. Survivin does not appear to be involved in the physiological regulation of apoptosis in adult colonic epithelium but is prominently expressed in CRC carcinoma. The mechanisms governing expression of survivin in malignant cells are presently unclear but a complex response to dedifferentiation of normal epithelium appears likely. Survivin mRNA expression was detected in a significantly greater proportion of CRC carcinoma than in normal mucosa samples (63.5% v 29.1%, respectively; p<0.001). In no case was survivin mRNA detected in normal tissue when the associated cancer was survivin negative. Approximately half the number of survivin positive tumors, but none of the survivin negative tumors, was associated with normal mucosa which also expressed this gene. These data appear at variance with previous studies which did not detect expression of either survivin mRNA by in situ hybridization or survivin protein by immuno-histochemistry in normal epithelium adjacent to tumors. Detection of mRNA transcripts by the more sensitive technique of RT-PCR20 suggests that survivin expression may represent an “intermediate” biological change identifying histologically normal mucosa at risk of neoplastic transformation.

Sarela et al. also reported that histologically normal CRC epithelial cells from patients with a history of CRC carcinoma are subject to as yet unidentified influences that result in significantly reduced apoptotic activity compared with that in similar cells from patients with no neoplasia. (Sarela et al., Gut 2000; 46:645-50)

Anti et al. presented data that showed severe imbalance of cell proliferation and apoptosis in the left colon and in the rectosigmoid colon in subjects with a history of large adenomas. The normal segment to segment pattern of proliferation was basically preserved in patients with large adenomas, although the rates of proliferation themselves were markedly higher than those found in average risk subjects. There was also a distinctive alteration in the distribution of proliferating cells within the colonic crypts, which was particularly marked in the left colon, and this change implied an additional risk for mutational events because highly vulnerable nondifferentiated cells at the mouth of the crypts were more exposed to intraluminal factors. Another interesting point was the relationship between changes in colonic epithelial cell kinetics and aberrant crypt foci (ACF), which are thought to be the microscopic precursors of both polyps and cancer. In CRC cancer patients, hundreds and hundreds of ACFs have been observed in the normal appearing mucosa of all segments of the colon. Only a few of these foci actually give rise to adenomas or carcinomas. The majority of these ACF's never reach macroscopic dimensions and many even regress. Nonetheless, their widespread distribution illustrates that in patients with CRC cancer, the normal appearing mucosa of the entire colon presents morphological changes with the potential to progress towards more advanced stages in the carcinogenetic pathway. The fact that an extensive tract of mucosa was found to harbor glands with a high proliferative propensity and reduced apoptotic activity, supports the “field cancerization” hypothesis, according to which longstanding exposure of the mucosa to carcinogens leads to diffuse biological abnormalities throughout large tracts of the colon, which can eventually produce cancers. It is also noteworthy that the severe imbalance between cell proliferation and apoptosis in the high risk group was localized in the left colon where most colon malignancies develop. (Anti et al., Gut 2001; 48:238-46)

Crowley-Weber et al. demonstrated that nicotine, a component of cigarette smoke, and sodium deoxycholate, NaDOC, a cytotoxic bile salt that increases in concentration in the gastrointestinal tract after a high fat meal, induce similar cellular stresses and that nicotine may enhance some of the NaDOC-induced stresses. Nicotine, at 0.8 mM, the very low sub-micromolar level occurring in the tissues of smokers: (1) increases oxidative stress; (2) activates NF-kB, a redox-sensitive transcription factor; (3) activates the 78 kD glucose regulated protein promoter, an indication of endoplasmic reticulum stress; (4) induces apoptosis; (5) enhances the ability of NaDOC to activate the 153 kD growth arrest and DNA damage promoter, an indication of increased genotoxic stress; and (6) enhances the ability of NaDOC to activate the xenobiotic response element. This leads to “field defects” in the mucosa of patients with colon carcinoma. (Crowley-Weber et al., Chem Biol Interact 2003; 145:53-66)

Garawal et al. described an early functional change characterizing field defects that seems to occur during progression to sporadic adenocarcinoma of the colon. This change was loss of the capacity to undergo apoptosis in response to damage. If a cell acquires mutations or epimutations that cause apoptosis resistance, this can lead to increased clonogenic survival and consequent clonal expansion. In addition, suppression of apoptosis leads to increased mutagenesis. Other factors that may have similar effects include smoking, dietary alcohol, low intake of calcium or antioxidant vitamins, and the nondietary factors of obesity and low physical activity. It was determined that resistance to the induction of apoptosis with a novel ex vivo bioassay, deoxycholate-induced apoptotic index (AI) was the most specific of the biomarkers and was present in 59% of the normal-appearing mucosal samples from patients with colon cancer. Apoptosis resistance highly correlated with a low level of differentiation, assessed with Dolichos bifloris agglutinin, DBA lectin staining. A high fraction (>35%) of tissue showing aberrant lectin staining (of nongoblet cells) was present only in the group of patients with colonic neoplasms, also making aberrant lectin reactivity a specific biomarker. The patchy nature of the field defects associated with apoptosis resistance and DBA staining underscores the necessity for multiple biopsies to assess colon cancer risk. The most important aspect of the study is the caveat of “patchiness”, because it probably applies to other biomarkers as well. Although ACF and microadenomas are expected as biomarkers of colon cancer risk when an entire animal colon is examined with methylene blue staining, such lesions are not easily detected in biopsy-sized tissue samples taken from human colons. The plant lectin DBA binds preferentially to terminal N-acetyl-galactosamine of mucin and that terminal N-acetyl-galactosamine occurred in well-differentiated goblet cell mucin of the upper colonic crypts. Based on this, DBA can be used as a probe to assess the level of differentiation of goblet cells. Sparse or aberrant DBA reactivity is a sensitive biomarker for colon cancer risk and a high level of aberrant lectin staining in nongoblet cells is specific for high-risk patients. Overall, the results of the study suggest that the live cell bioassay for AI and the more practical DBA staining assay on preserved tissue samples are promising biomarkers of colon cancer risk, but multiple samples must be obtained to give a valid indication of risk. (Bernstein et al., Ann Surg Oncol 2002; 9:505-17)

Chen et al. compared gene expression levels in morphologically normal-appearing colon mucosa from cancer patients with those in mucosa from patients without cancer. One set of data, consisted of samples from patients with cancer in the recto-sigmoid colon, the other set was from patients with cancer in the ascending colon. In both studies, the values obtained from the cancer patients were highly variable, much more so than the corresponding values from the controls. This finding parallels the observations made in APCmin and wild-type mice, although the variation in humans was even higher. Despite the great variability, expression levels for several genes were much higher in some samples from cancer patients than for any samples from controls. For example, four of the genes that were significantly up-regulated in normal-appearing mucosa of APCmin mice—CXCR2, GRO-α, COX-2, and OPN—were upregulated in normal-appearing mucosa from some cancer patients to levels 50-200 times greater than those in controls. In addition, in some cancer patients, PPAR-α, -δ, and -γ were down-regulated 50-100-fold. Seven genes were significantly up-regulated in morphologically normal mucosa from patients with recto-sigmoid cancer relative to controls: MCSF-1, OPN, IL-8, COX-2, CXCR2, p21, and CD44. Two genes-PPAR-δ, and -γ were significantly down-regulated. Similar results were obtained for the ascending colon. Six of the seven genes significantly up-regulated in recto-sigmoid mucosa were also up-regulated in the ascending colon, MCSF-1, OPN, IL-8, COX-2, CXCR2, and CD44 along with COX-1. Likewise, PPAR-δ, and -γ were significantly down-regulated in the ascending colon. The difference between cancer patients and controls was even more striking when the relative expression levels of three of the most up-regulated genes, COX-2, OPN, and MCSF-1, were considered together. In virtually every sample from a cancer patient, at least one of these three genes was significantly up-regulated relative to its expression level in any sample from a control. This analysis suggested that expression levels of these three genes considered together may be sufficient to distinguish normal colon mucosa in colon cancer patients from colon mucosa in controls. (Chen et al., Cancer Res 2004; 64:3694-700)

Alterations in signaling pathways of FGFR, EGFR, ERBB2, and PI3K were detected in nearly two-thirds of breast and colorectal tumors that were comprehensively examined in a recent report from the Vogelstein laboratory. The complexity of genetic analysis of CRC was further discussed in the report in which the authors stated that “these genetic analyses can only identify candidate genes that may play a role in cancer and do not definitively implicate any gene in the neoplastic process”. (Leary et al., Proc Natl Acad Sci USA 2008; 105:16224-9)

These studies demonstrate a wide spectrum of genetic and protein analyses of CRC and histologically normal tissue surrounding CRC tumors. However, there are few duplications of genes and proteins between the various authors. Preliminary work in this laboratory suggested that mass spectrometry of the CRC and normal satellite tissue could identify specific reproducible protein biomarkers that would characterize tissue at risk for CRC. Specifically, MALDI imaging, IMS, of tissue and liquid chromatography (electrospray) mass spectrometry, LCMS, of tissue protein extracts. The goal would be to use these putative proteins as the basis for a screening test of stool.

Recently in specimens from two consecutive patients, imaging MALDI (IMS) demonstrated chemical evidence of two proteins, gi|119592539 hCG1787564 [Homo sapiens] Mass: 57590, and gi|119592490 hCG2040674 [Homo sapiens] Mass: 108178 in colon adenocarcinoma and in histopathologically normal satellite tissue. (Pevsner et al., British Mass Spectrometry Society; 2007) This finding represents a potential marker for field cancerization or a field defect, e.g., age-related hypermethylation in normal colonic mucosa. Such alterations result in microsatellite instability, and synchronous and metachronous lesions which develop into cancers. (Young et al., Cancer Epidemiol Biomarkers Prev. 2006; 15:1778-1784) Therefore, histopathology alone may significantly affect therapy by underestimating the extent of metaplastic or malignant disease. It would be useful to supplement conventional histopathology with, for instance, the addition of proteomic classification with LCMS, and imaging MALDI, IMS that can better identify the extent of metaplastic disease beyond the recognized tumor.

### SUMMARY OF THE INVENTION

The present invention provides a method of diagnosing carcinoma, for instance, colon, rectal or colorectal carcinoma, by determining whether one or more carcinoma associated markers is present in a sample. The carcinoma associated marker may be, for instance, any one presented in FIG. 1. One, two, three, four, five, six, seven, eight, ten, twelve or more of the carcinoma associated markers, such as those presented in FIG. 1, may be present in the sample and may be identified. In some embodiments it is the abundance of one or more carcinoma associated markers that is predictive of the likelihood of carcinoma or the likelihood that a biological sample contains cancerous cells. Likewise, the relative abundance of one or more carcinoma marker may be predictive of the relative stage of carcinoma.

The methods may optionally include quantifying one or more of the carcinoma associated markers such as those presented in FIG. 1. The methods may further include comparing the amount of one or more carcinoma associated markers in the sample with either (i) the amount determined for normal samples known to be substantially free of cancer cells or (ii) the amount determined for cells that are known to be non-cancerous. The relative absence of one or more of the carcinoma associated markers such as those presented in FIG. 1 in the sample may indicate that the likelihood that cells are cancerous is relatively low.

The present invention also provides methods for defining the limits of normal tissue and the extent of cancerous tissue. Likewise, the present invention features methods for defining the field that should be resected surgically. In these aspects, multiple biological samples may be obtained from a relatively small area anatomically. The methods may include comparing the amount of one or more carcinoma associated marker in one sample with either (i) the amount determined for normal samples known to be substantially free from cancer cells or (ii) the amount determined for cells that are known to be non-cancerous. The relative absence of one or more of the carcinoma associated marker such as those presented in FIG. 1 in the sample may indicate that the likelihood that cells are cancerous is relatively low. The relative abundance of one or more of the carcinoma associated marker such as those presented in FIG. 1 in the sample may indicate that the likelihood that cells are cancerous is relatively high. Using such information, an informed decision may be made regarding the boundaries that should be resected in a surgical resection of cancerous tissue.

The present invention further provides methods for providing a prognosis in a subject. In some embodiments, the present invention features methods for predicting the likely outcome of a cancer comprising determining the presence or absence of one or more carcinoma associated marker such as those presented in FIG. 1 in a sample. One, two, three, four, five, six, seven, eight, ten, twelve or more of the carcinoma associated markers such as those presented in FIG. 1 may be present in the sample and may be identified. One, two, three, or more of the carcinoma associated markers may be present in a tissue sample and may be elevated in comparison to samples obtained from normal non-cancerous tissue. The relative abundance of one or more carcinoma associated marker may be compared to values obtained from samples known to contain no cancerous cells to values representative of one or more subjects known to have cancer at a particular stage.

The present invention also provides methods for determining the likelihood that cells in a biological sample may become cancerous by determining the presence of or relative abundance of one or more carcinoma associated marker in the biological sample compared to a sample known to contain no cancerous cells or to a value known to be representative of biological samples containing substantially no cancerous cells. These methods are especially applicable to, for instance, colon, rectal or colorectal tissue. The carcinoma associated marker may be any one presented in FIG. 1. One, two, three, four, five, six, seven, eight, ten, twelve or more of the carcinoma associated markers such as those presented in FIG. 1 may be present in the sample and may be identified. In some embodiments it is the abundance of one or more carcinoma associated markers that is predictive of the likelihood of cells becoming cancerous. Likewise, the relative abundance of one or more carcinoma marker may be predictive of the relative stage of carcinoma or of benign cell changes. Such benign cell changes may be predictive of the likelihood that a cell will become cancerous.

The methods may optionally include quantifying one or more of the carcinoma associated markers such as those presented in FIG. 1. The methods may further include comparing the amount of one or more carcinoma associated markers in the sample with either (i) the amount determined for normal samples known to be substantially free from cancer cells or (ii) the amount determined for cells that are known to be non-cancerous. The relative absence of one or more of the carcinoma associated markers such as those presented in FIG. 1 in the sample indicates that the likelihood that cells are cancerous is relatively low.

The sample used for determining the presence of one or more carcinoma associated markers may be an in vivo tissue sample. Any size suitable for obtaining histologically and for providing sufficient cells for analysis may be used. The tissue sample may be obtained from any organ or body part, such as, for instance, a biopsy, such as biopsy of a colon polyp. The one or more carcinoma associated markers such as a those presented in FIG. 1 may be identified by many methods well known to those of skill in the art including Matrix assisted laser desorption ionization (MALDI) mass spectrometry. The carcinoma associated markers may also be identified by contacting the sample with an antibody that specifically binds to the carcinoma associated marker under conditions permitting formation of a complex between the antibody and the carcinoma associated marker, and optionally measuring the amount of complexes formed, thereby determining the amount of the carcinoma associated marker in the sample.

The invention also provides methods for diagnosing cancer or for assessing the risk that a tissue or cell may become cancerous comprising the steps of: (a) obtaining a biological sample from a patient; (b) measuring an amount of one or more carcinoma associated markers present in the biological sample; and (c) comparing the amount of one or more carcinoma associated marker with a predetermined value, whereby the amount of carcinoma associated markers relative to the predetermined value indicates the relative likelihood of a biological sample containing cancerous cells or of a biological sample containing cells that in the future will become cancerous. The predetermined value may represent the amount of the carcinoma associated marker present in a sample of tissue that is known to be non-cancerous or to contain substantially no cancer cells.

Further, the present invention provides a diagnostic kit for diagnosing carcinoma or for assessing the risk of carcinoma development. The kit contains one or more reagents, such as, for instance one or more antibodies to one or more carcinoma associated markers useful for determining the presence or absence of one or more carcinoma associated markers such those presented in FIG. 1 in a biological sample. The kit may further contain instructions and one or more printed values or ranges of values that represent the amount of one or more carcinoma associated markers present in a normal biological sample that does not contain cancerous cells, the amount of one or more carcinoma associated markers present in a biological sample that does contain cancerous cells, the amount of one or more carcinoma associated markers present in a representative sample obtained from at least 10 or more subjects.

### BRIEF DESCRIPTION OF THE FIGURES

FIG. 1 provides useful carcinoma associated markers. The exemplified proteins were isolated from colon polyp biopsies from three patients.

FIG. 2 represents a MALDI TOF spectrum of colorectal adenocarcinoma and satellite normal tissue. A spectrum obtained from the tumor, and satellite tissue was analyzed with Mascot software. The search criteria included only one missed cleavage. Peptide mass fingerprinting, PMF, from the IMS image demonstrated the exact same two proteins, gi|119592539 hCG1787564 [Homo sapiens] Mass: 57590, and gi|119592490 hCG2040674 [Homo sapiens] Mass: 108178 in the colon adenocarcinoma and in the histopathologically normal satellite tissue.

FIG. 3 is a histologic section of a colorectal carcinoma tumor stained with hematoxylin and eosin, 1μ thick, micrograph 40×. The arrowheads outline the edge of the colon adenocarcinoma.

FIG. 4 is a MALDI image. The right outlined area corresponds to the tumor in the histology section, and the smaller left outlined region corresponds to the histologically normal satellite tissue. The identical hCG isoforms were identified in both loci. The smaller locus represents a satellite “field defect” with potential for cancerization.

FIG. 5 is a hematoxylin and eosin stained histologic section of histopathologically benign appearing colon polyp. The central fibrotic contracted zone is shown by opposing arrows. An artery at the lower growth margin of the polyp infiltrating normal tissue is identified by the lower arrow. Tissue section micrograph 40×.

FIG. 6 represents a contiguous section to the H & E histolologic section in FIG. 5 that was applied to a MALDI conductive plate. The section was covered with a Sinapic acid matrix by sublimation. Tissue section micrograph 40×.

FIG. 7 represents a MALDI image of a polyp revealing tentative protein identifications (obtained by query of the National Center for Bioinformatics Not Repeatable database with the mass of the proteins) that are visible at the transition zone between the polyp and the histologically normal peripheral tissue.

FIG. 8 represents a MALDI image of a polyp revealing tentative protein identifications (obtained by query of the NCBInr database with the mass of the proteins) that are visible at the transition zone between the polyp and the histologically normal peripheral tissue.

FIG. 9 represents a MALDI image of a polyp revealing tentative protein identifications (obtained by query of the NCBInr database with the mass of the proteins) that are visible at the transition zone between the polyp and the histologically normal peripheral tissue.

FIG. 10 represents a MALDI image of a polyp revealing tentative protein identifications (obtained by query of the NCBInr database with the mass of the proteins) that are visible at the transition zone between the polyp and the histologically normal peripheral tissue.

FIG. 11 represents a MSMS mass spectroscopy spectra of alcohol dehydrogenase 1B GI 113394.

FIG. 12 represents a MSMS mass spectroscopy spectra (MALDI TOF TOF) of GI 229751, Chain A, alpha-ferrous-carbonmonoxy, beta-cobaltous-deoxy hemoglobin (T state).

FIG. 13 represents a MSMS mass spectroscopy spectra (MALDI TOF TOF) of galectin 4 GI 5453712.

FIG. 14 represents a MSMS mass spectroscopy spectra (MALDI TOF TOF) of vimentin GI 37852.

FIG. 15 represents a MSMS mass spectroscopy spectra (MALDI TOF TOF) of hemoglobin beta GI 40886941.

### DETAILED DESCRIPTION OF THE INVENTION

As used herein, the following terms mean as follows:

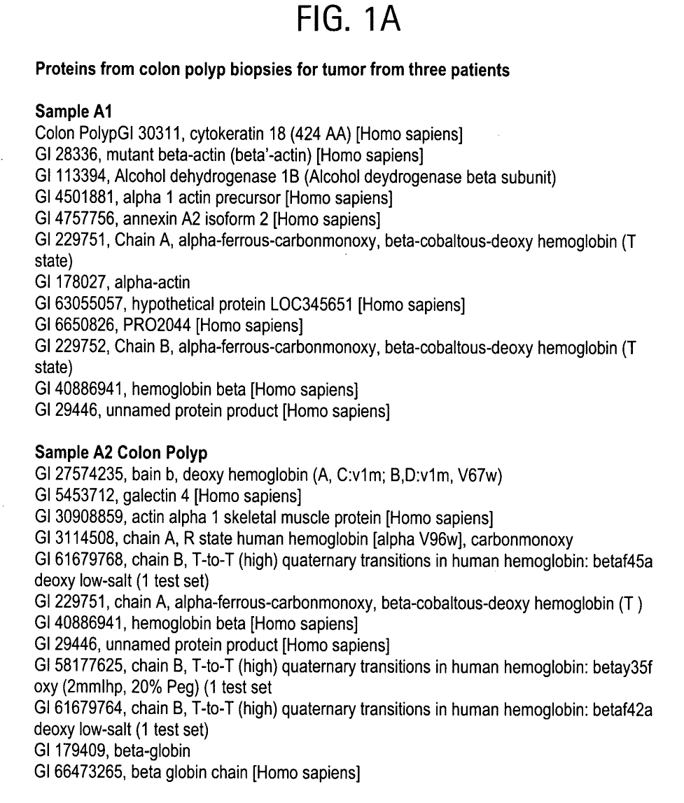

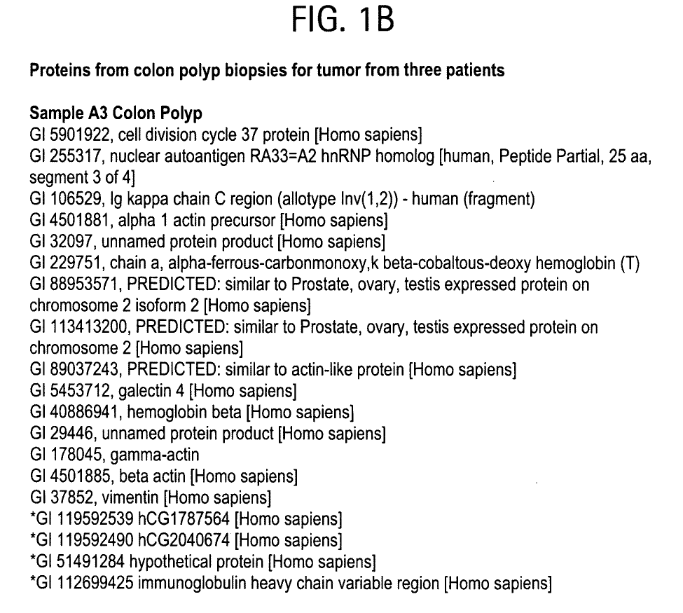

As used herein, “carcinoma associated marker” means any molecule, such as a protein, peptide or fragment thereof whose presence, absence or amount in absolute quantity or in quantity relative to other molecules may be used as evidence of whether a tissue contains cancerous cells or whether a cell is cancerous. Stated differently, a carcinoma associated marker is any molecule that may be used as a statistically significant predictor of the presence or absence of cancerous cells. The carcinoma associated marker may be normally absent altogether from non-cancer tissue, or it may be present in cancer tissue in an amount that is either more than or less than the amount of the carcinoma associated marker found in non-cancer tissue. The “carcinoma associated markers presented in FIG. 1” include, for instance, GI 30311, cytokeratin 18 (424 AA) [Homo sapiens], GI 28336, mutant beta-actin (beta'-actin) [Homo sapiens], GI 113394, Alcohol dehydrogenase 1B (Alcohol deydrogenase beta subunit), GI 4501881, alpha 1 actin precursor [Homo sapiens], GI 4757756, annexin A2 isoform 2 [Homo sapiens], GI 229751, Chain A, alpha-ferrous-carbonmonoxy, beta-cobaltous-deoxy hemoglobin (T state)GI 178027, alpha-actinGI 63055057, hypothetical protein LOC345651 [Homo sapiens]GI 6650826, PRO2044 [Homo sapiens], GI 229752, Chain B, alpha-ferrous-carbonmonoxy, beta-cobaltous-deoxy hemoglobin (T state), GI 40886941, hemoglobin beta [Homo sapiens], GI 29446, unnamed protein product [Homo sapiens], GI 27574235, bain b, deoxy hemoglobin (A, C:v1m; B,D:v1m, V67w), GI 5453712, galectin 4 [Homo sapiens], GI 30908859, actin alpha 1 skeletal muscle protein [Homo sapiens], GI 3114508, chain A, R state human hemoglobin [alpha V96w], carbonmonoxyGI 61679768, chain B, T-to-T (high) quaternary transitions in human hemoglobin: betaf45a deoxy low-salt (1 test set), GI 229751, chain A, alpha-ferrous-carbonmonoxy, beta-cobaltous-deoxy hemoglobin (T), GI 40886941, hemoglobin beta [Homo sapiens], GI 29446, unnamed protein product [Homo sapiens], GI 58177625, chain B, T-to-T (high) quaternary transitions in human hemoglobin: betay35f oxy (2 mmIhp, 20% Peg) (1 test setGI 61679764, chain B, T-to-T (high) quaternary transitions in human hemoglobin: betaf42a deoxy low-salt (1 test set), GI 179409, beta-globin, GI 66473265, beta globin chain [Homo sapiens], GI 5901922, cell division cycle 37 protein [Homo sapiens], GI 255317, nuclear autoantigen RA33=A2 hnRNP homolog [human, Peptide Partial, 25 aa, segment 3 of 4] GI 106529, Ig kappa chain C region (allotype Inv(1,2))-human (fragment) GI 4501881, alpha 1 actin precursor [Homo sapiens], GI 32097, unnamed protein product [Homo sapiens], GI 229751, chain a, alpha-ferrous-carbonmonoxy, k beta-cobaltous-deoxy hemoglobin (T), GI 88953571, PREDICTED: similar to Prostate, ovary, testis expressed protein on chromosome 2 isoform 2 [Homo sapiens], GI 113413200, PREDICTED: similar to Prostate, ovary, testis expressed protein on chromosome 2 [Homo sapiens], GI 89037243, PREDICTED: similar to actin-like protein [Homo sapiens], GI 5453712, galectin 4 [Homo sapiens], GI 40886941, hemoglobin beta [Homo sapiens], GI 29446, unnamed protein product [Homo sapiens], GI 178045, gamma-actin, GI 4501885, beta actin [Homo sapiens], and GI 37852, vimentin [Homo sapiens].

Other preferred “carcinoma associated markers” include GI 229751, chain A, alpha-ferrous-carbonmonoxy, beta-cobaltous-deoxy hemoglobin (T), GI 40886941, hemoglobin beta [Homo sapiens], GI 5453712, galectin 4 [Homo sapiens] 50×, GI 113394, alcohol deydrogenase beta subunit, GI 40886941, hemoglobin beta [Homo sapiens], GI 5453712, galectin 4 [Homo sapiens] (50× lower in colorectal cancer than normal colon), GI 37852, vimentin [Homo sapiens], GI 119592539 hCG1787564 [Homo sapiens], GI 119592490 hCG2040674 [Homo sapiens], GI 51491284, and GI 112699425 immunoglobulin heavy chain variable region [Homo sapiens].

As used herein, a “predetermined value” is a standardized value based on a control. For example, a predetermined value can be based on an amount of carcinoma associated marker such as those presented in FIG. 1 or other preferred carcinoma associated markers that are present in a biological sample obtained from a tissue sample that is known to contain cancerous cells or that is known to contain no cancerous cells.

The term “amount” is used within the context of the analytical method used to measure the different carcinoma associated markers such as those presented in FIG. 1 and may reflect a number, a concentration, etc., depending upon the analytical method chosen to measure the carcinoma associated markers such as those presented in FIG. 1.

The term “relative amount” refers to the amount of one or more carcinoma associated marker present in a biological sample in proportion to the amount of the same one or more carcinoma associated markers present in a corresponding biological sample known to contain no or substantially no cancerous tissue or cancer cells.

The term “biological sample,” as used herein, generally refers to urine, saliva, serum, plasma, and especially tissue samples containing whole cells.

The term “detecting” as used herein refers to identifying the presence of, identifying the presence of in relative amounts relative to another molecule or carcinoma associated markers such as those presented in FIG. 1 relative to a predetermined value, or quantifying in absolute amounts.

Carcinoma associated markers can be directly measured, for example, using anti-marker antibodies in an immunoassay, such as a Western blot or ELISA. Carcinoma associated markers can be indirectly measured, for example, using a capture antibody that binds the carcinoma associated marker.

The methods of the invention can be used alone or in combination with any known test for determining the presence of or diagnosing carcinoma or for determining the prognosis of carcinoma, including, but not limited to, X-Ray, ultrasound, CAT scan, and other blood marker analysis. The methods of the invention can be used to screen a biological sample collected at any time either before or after a first diagnosis of carcinoma has been made.

The amount of carcinoma associated markers in a biological sample can be determined using any method known in the art, including, but not limited to, Matrix-assisted laser desorption/ionization (MALDI), immunoassays using antibodies specific for the carcinoma associated marker. Any assay that functions to qualitatively or quantitatively determine variations in sample concentrations of carcinoma associated markers from normal levels can be employed in the practice of the invention.

For example, a monoclonal anti-carcinoma associated marker antibody can be generated by immunizing a mouse with the carcinoma associated marker. Once an immune response is detected, e.g., antibodies specific for the carcinoma associated marker are detected in the mouse serum, the mouse spleen is harvested and splenocytes are isolated. The splenocytes are then fused by well-known techniques to any suitable myeloma cells, for example, cells from cell line SP20 available from the American Type Culture Collection (ATCC). Hybridomas are selected and cloned by limited dilution. The hybridoma clones are then assayed by methods known in the art for cells that secrete antibodies capable of binding the carcinoma associated marker. Ascites fluid, which generally contains high levels of antibodies, can be generated by immunizing mice with positive hybridoma clones.

Any type of fusion phage, monoclonal, or polyclonal antibodies can be used in immunoassays of the invention, so long as the antibodies can be used in a reproducible fashion as markers for various carcinoma associated markers or as measures of the different levels of carcinoma associated markers observed in normal and variant populations.

In one embodiment, an amount of carcinoma associated marker can be measured using a capture antibody followed by a labeled secondary antibody using a strategy as described, for example, in U.S. Pat. No. 6,429,018, herein incorporated by reference. The label on the secondary antibody can comprise any chemical, radioactive, lanthanide, colored dye, or genetic tag used in enzyme-linked immunosorbent assays (ELISAs), Western blots, and other sensitive and specific immunoassays and immunoradiometric assays using known methodology. These include conjugating the antibody with horseradish peroxidase or alkaline phosphatase that are easily measurable, typically using colorimetric, fluorometric or luminescent substrates. Genetic labels include firefly luciferase, employed because luciferase produces a bioluminescent molecule when incubated with its substrate, luciferin.

Matrix-Assisted Laser Desorption/Ionization (MALDI).

Matrix-assisted laser desorption/ionization (MALDI) is a soft ionization technique used in mass spectrometry, allowing the analysis of biomolecules (biopolymers such as proteins, peptides and sugars) and large organic molecules (such as polymers, dendrimers and other macromolecules), which tend to be fragile and fragment when ionized by more conventional ionization methods. It is most similar in character to electrospray ionization both in relative softness and the ions produced (although it causes many fewer multiply charged ions). The ionization is triggered by a laser beam (normally a nitrogen laser). A matrix is used to protect the biomolecule from being destroyed by direct laser beam and to facilitate vaporization and ionization.

The matrix consists of crystallized molecules, of which the three most commonly used are 3,5-dimethoxy-4-hydroxycinnamic acid (sinapinic acid), α-cyano-4-hydroxycinnamic acid (alpha-cyano or alpha-matrix) and 2,5-dihydroxybenzoic acid (DHB). A solution of one of these molecules is made, often in a mixture of highly purified water and an organic solvent (normally acetonitrile (ACN) or ethanol). Trifluoroacetic acid (TFA) may also be added. A good example of a matrix-solution would be 20 mg/mL sinapinic acid in ACN:water:TFA (50:50:0.1). The identity of suitable matrix compounds is determined to some extent by trial and error, but they are based on some specific molecular design considerations: they have a low molecular weight; they are acidic and act as a proton source to encourage ionization of the analyte; they have a strong optical absorption in the UV and efficiently absorb the laser irradiation; and they are functionalized with polar groups allowing use in aqueous solutions.

The matrix solution is mixed with the analyte (e.g. protein sample). The organic solvent allows hydrophobic molecules to dissolve into the solution, while the water allows for water-soluble (hydrophilic) molecules to do the same. This solution is spotted onto a MALDI plate (usually a metal plate designed for this purpose). The solvents vaporize, leaving only the recrystallized matrix, but now with analyte molecules spread throughout the crystals. The matrix and the analyte are said to be co-crystallized in a MALDI spot.

The laser is fired at the crystals in the MALDI spot. The matrix absorbs the laser energy, and the matrix is ionized by this event. The matrix transfers part of its charge to the analyte molecules (e.g. protein) thereby ionizing them while still protecting them from the disruptive energy of the laser. Ions observed after this process consist of a neutral molecule [M] and an added or removed ion. Together, they form a quasimolecular ion, for example [M+H]⁺ in the case of an added proton, [M+Na]⁺ in the case of an added sodium ion, or [M−H]⁻ in the case of a removed proton. MALDI is capable of creating singly-charged ions, but multiply charged ions ([M+nH]ⁿ⁺) can also be created with electrospray. Note that these are all even, odd, or multiple charged electron species. Ion signals of radical cations can be observed eg. in case of matrix molecules and other stable molecules.

Atmospheric pressure (AP) matrix-assisted laser desorption/ionization (MALDI) is an ionization technique (ion source) that in contrast to vacuum MALDI operates at normal atmospheric environment. In vacuum MALDI, ions are typically produced at 10 mTorr or less while in AP-MALDI ions are formed in atmospheric pressure.

AP-MALDI is used in mass spectrometry (MS) in a variety of applications including proteomics and drug discovery fields. AP-MALDI mass spectrometry is often used in proteomics, DNA/RNA/PNA, lipids, oligosaccharides, phosphopeptides, bacteria, small molecules and synthetic polymers, similar applications as available also for vacuum MALDI instruments.

The AP-MALDI ion source is easily coupled to an ion trap mass spectrometer or any other MS system equipped with ESI (electrospray ionization) or nanoESI source.

The type of a mass spectrometer most widely used with MALDI is the TOF (time-of-flight mass spectrometer) because of its large mass range. The TOF measurement procedure is suited to the MALDI ionization process since the pulsed laser takes individual ‘shots’ rather than working in continuous operation. MALDI-TOF instruments are typically equipped with an “ion mirror,” deflecting ions with an electric field thereby doubling the ion flight path and increasing the resolution. Today, commercial reflectron TOF instruments reach a resolving power m/m of well above 20'000 FWHM (full-width half-maximum, m defined as the peak width at 50% of peak height).

In proteomics, MALDI is used for identifying proteins isolated through gel electrophoresis: SDS-PAGE, size exclusion chromatography, and two-dimensional gel electrophoresis. One method used is peptide mass fingerprinting by MALDI-MS, or with post ionization decay or collision-induced dissociation.

IMS.

The history of IMS (Imaging Maldi Spectroscopy) began with single cell studies of Aplysia californica neurons by matrix-assisted laser desorption/ionization time-of flight mass spectrometry (MALDI/TOF-MS). This was probably the first direct tissue MALDI identification of peptides and tissue profiling based on ion density. (Garden et al., J Mass Spectrom 1996; 31:1126-30) A refinement of this technique, imaging MALDI was described in human buccal mucosa, and rat pituitary and pancreas glands using two different approaches: direct targeting of the tissue itself and by analysis of blotted targets previously exposed to the tissue. (Caprioli et al., Anal Chem 1997; 69:4751-60) An “MS Image Tool,” using the peptide neurotensin (peak at m/z 1674) significantly improved the data acquisition, speed of IMS, and utilization of the technique. (Stoeckli et al., J Am Soc Mass Spectrom 1999; 10:67-71) In a laser, capture microdissection (LCM) study of both tumor and normal human breast tissue fixed in ethyl alcohol, and stained with hematoxylin and eosin, normal tissue, carcinoma in situ, invasive carcinoma, and metastatic carcinoma could be distinguished by their different MALDI spectra. The introduction of “BioMap” software, by applying a baseline correction to the spectra and integrating over the peak of interest, demonstrated mouse brain images of amyloid β, and Aβ peptides. That report was proof of the principle that MALDI images of tissue could be obtained based upon the mass spectrometry mass/charge, m/z peak of interest. (Stoeckli et al., Nat Med 2001; 7:493-6) Subsequent profiling and IMS of normal mouse epididymis identified different protein activity (ion densities) throughout the sections. (Chaurand et al., Electrophoresis 2002; 23:3125-35)

The negative effects on IMS resolution from destructive tissue freezing artifacts, excessive dehydration due to ethanol fixation, paraformaldeyde cationization, embedding artifacts from OCT polymer and agar, and coarse matrix crystal size were first described in a report on spatial profiling of invertebrate ganglia. (Kruse et al., J Am Soc Mass Spectrom 2003; 14:752-9) In a follow-up report, they suggested a solution to the problem of freezing artifacts using glycerol and the related compounds ethane-1,2-diol and propane-1,2-diol to stabilize cellular membranes. (Rubakhin et al., Anal Chem 2003; 75:5374-80) A subsequent report suggested direct liquid nitrogen immersion of tissue in aluminum wrapping as a means of rapid fixation, but ignored the known consequences of freezer artifact. (Schwartz et al., J Mass Spectrom 2003; 38:699-708) Adjunctive histologic staining with methylene blue stained tissues on standard metal plates or indium-tin coated glass slides were shown to be compatible with IMS, but cresyl violet stain decreased IMS signal intensity. (Chaurand et al., Anal Chem 2004; 76:1145-55)

Tissue blotting with trypsin digestion for MALDI TOF data base analysis was shown to be useful in analyte localization, but was destructive to tissue morphology. (Bunch et al., Rapid Communications in Mass Spectrometry 2004; 18:3051-60) Others described a less destructive trypsin digest step to their prior tissue blotting technique for IMS. (Rohner et al., Mech Ageing Dev 2005; 126:177-85) Another method, matrix-enhanced secondary ion mass spectrometry (ME) SIMS was described and used for direct molecular imaging of the ganglia of the freshwater snail, Lymnaea stagnalis. (Altelaar et al., Anal Chem 2005; 77:735-41) However, this technique presented significant limitations and was proved unsuitable for direct tissue imaging. A refinement of IMS, oversampling with complete sample ablation at each sample position on the target plate, provided significant resolution enhancement with a translation stage raster step size of 25 μM. A 40 μM object could now be resolved with a 100 μM laser. (Jurchen et al., Journal of the American Society for Mass Spectrometry 2005; 16:1654-9)

Tissue treatments with organic solvents such as chloroform, acetone, hexane, toluene, or xylene were shown to be an effective and rapid method for signal enhancement in MALDI direct tissue profiling. (Lemaire et al., Analytical Chemistry 2006; 78:7145-53) These studies demonstrated that solvent treatments partially removed lipids from the tissue surface. Compared to previous studies with ethanol, chloroform/xylene solvent, rinsing is more specific for lipid removal and does not generate delocalization or extraction of most soluble peptides/proteins as tested by immuno-histochemistry experiments. Among all the tested solvents, chloroform and xylene produced the greatest increase in MALDI signal intensity and number of detected peptides/proteins. However, this treatment does not reduce salt adducts as does alcohol treatment. The results suggest that it is possible to detect, after organic rinsing treatments, compounds, such as peptides/proteins present in the cytoplasm, that were masked by lipids in the tissue.

Clench et al. reported the development and application of a method using 9-aminoacridine as a matrix for negatively charged ions in MALDI imaging. (Burrell et al., J Exp Bot 2007; 58:757-63) Crossman demonstrated the need for thin sections to avoid differential extraction efficiency of matrix solvent in different tissues. (Crossman et al., Rapid Communications in Mass Spectrometry 2006; 20:284-90) Pevsner demonstrated direct cellular MALDI identification of proteins in fixed cells and tissues without freezer artifact, tissue corrosion by matrix solvents or the use of tissue blotting. This was confirmed in a later report. (Pevsner et al., Direct identification of proteins from cells and tissues using MALDI TOF. Anal Chem.; Pevsner et al., J Soc Gynecol Investig 2006; 13:A1-B10; Groseclose et al., J Mass Spectrom 2007; 42:254-62)

Metal-assisted (MetA) secondary ion mass spectrometry (SIMS), a variation on SIMS as well as matrix-assisted laser desorption/ionization (MALDI) IMS can provide images from tissue, but the duration of these protocols were highly dependent on sample size and technique parameters. The duration of these studies averaged approximately 5 h. (Altelaar et al., Nat Protoc 2007; 2:1185-96)

Agar et al. studied multiple solvent/matrix combinations. However, the tissue sections at the electron microscopic level demonstrated both freezing artifact and structural distortion, indicating that their method disrupts normal subcellular structures such as mitochondria. (Agar et al., Matrix Solution Fixation: Histology-Compatible Tissue Preparation for MALDI Mass Spectrometry Imaging. Anal Chem, Published on World Wide Web Sep. 7, 2007) Baluya et al reported a variation on inkjet-printed matrix application to tissue specimens previously described by Sloane et al. (Baluya et al., Anal Chem 2007; 79:6862-7; Sloane et al., Mol Cell Proteomics 2002; 1:490-9) The matrix application was of better quality and more reproducible than from specimens prepared by the electrospray and airbrush methods, but still was not completely uniform. A uniform method of tissue matrix application is sublimation. Sublimation is solvent free, rapid, and was successfully used to identify lipids in brain tissue, and more recently described for proteins or peptides. The challenge of IMS in formalin fixed paraffin embedded tissue was first addressed by and then by Pevsner and later by Stauber. (Hankin et al., J Am Soc Mass Spectrom 2007; 18:1646-52; Pevsner et al. “Microtubule Associated Proteins (MAP) and Motor Molecules Direct Tissue MALDI Identification and Imaging,” 2007; Pevsner et al., 2007, British Mass Spectrometry Society, Edinburgh, Scotland “Colon Cancer: Protein Biomarkers in Tissue and Body”; Pevsner et al., “Colorectal Carcinoma—Field Defects in Satellite Tissue.” 2007, British Mass Spectrometry Society, Edinburgh, Scotland, Robinson College, Cambridge, UK; Puolitaival et al., J Am Soc Mass Spectrom 2008; 19:882-6; Lemaire et al., J Proteome Res 2007; 6:1295-305; 48. Stauber et al., J Proteome Res 2008; 7:969-78)

This invention is illustrated in the experimental section that follows. These examples are set forth to aid in understanding the invention but are not intended to, and should not be construed to, limit in any way the invention as set forth in the claims.

### Example 1

Specimens from individuals with colon adenocarcinoma were compared with normal appearing colonic tissue from average risk individuals who completed a screening colonoscopy. During endoscopy using standard biopsy forceps, eight samples each measuring 2 mm×3 mm were obtained from each patient. Samples were immersed immediately in a solution of dimethyl sulfoxide (DMSO) 2%, glycerol 20%, and ethyl alcohol 78% and stored at 4° C. This mixture both fixes without cross-linking the proteins and cryoprotects the tissue. (Terracio et al., J Histochem Cytochem. 1981; 29:1021-1028; Rosene et al., J Histochem Cytochem. 1986; 34:1301-1315) Cryosections were obtained for IMS, hematoxylin and eosin histopathological staining, and protein extraction. A pathologist blinded to the clinical data, reviewed each specimen. Proteins were extracted from the colon tissue with organic solvent and high pressure using ProteoSolve and the Barocycler respectively (Pressure BioSciences, West Bridgewater, Mass.). The protein fraction was trypsinized, and the peptides studied with LCMS (Hitachi NanoFrontier nLC, Dallas, Tex.).

Additional proteins were identified in the colon biopsies using the Mascot search engine interrogation of the NCBInr and Swiss prot databases. These proteins are presented in FIG. 1 with NCBInr assigned GI numbers. The combination of high pressure protein extraction with an organic solvent and LCMS significantly increased the yield of peptides obtained from the tissue digest and identification of heretofore unidentified proteins. The mass numbers of these proteins are used to reconstruct the IMS images, and localize the proteins in the tissue (images not shown).

The presence of variations in polyp proteins in the biopsy tissue demonstrates genetic field differences that are predictive of carcinoma not only developing in these polyps, but at other sites in the colon as synchronous and metachronous lesions. These findings alter the current paradigm of histopathology tissue diagnosis for tumor and require examining the biopsy tissue with histopathology and mass spectrometry for complete diagnosis.

### Example 2

Biopsy tissue was cryo protected by immediate immersion in a mixture of dimethyl sulfoxide, DMSO 2%/glycerol 20%/ethanol 78%. Cryo sections, 1μ thick, could be obtained without freezer artifact that would otherwise destroy the tissue architecture. Contiguous 1μ sections were obtained for histology, IMS, and protein extraction with high pressure (Barocycler, Pressure BioSciences). The complete protocols are detailed in the references, herein incorporated by reference. (Pevsner et al. “Microtubule Associated Proteins (MAP) and Motor Molecules: Direct Tissue MALDI Identification and Imaging.” 2007, British Mass Spectrometry Society, Edinburgh, Scotland ; Pevsner et al., “Colon Cancer: Protein Biomarkers in Tissue and Body” 2007; Pevsner et al. British Mass Spectrometry Society, Edinburgh, Scotland, “Colorectal Carcinoma—Field Defects in Satellite Tissue” 2007; Vecchione et al., “Prophylactic Estrogen (Estradiol) Therapy” 2007, British Mass Spectrometry Society, Robinson College, Cambridge, UK; Remsen et al., “IMAGING MALDI of Colorectal Carcinoma—Field Defects in Satellite Tissue” 2008; Pevsner et al. 2007, British Mass Spectrometry Society, Edinburgh, Scotland, Robinson College, Cambridge, UK, “Mass Spectrometry of Buccal Mucosa -Biomarkers for Biodosimetry in Radiation Incidents”; 2008, Armed Forces Radiobiology Research Institute, Bethesda, Md., Bethesda, Md.).

Trypsin 1 μg/μL in 100 mM ammonium bicarbonate, pH 8, was applied to the tissue for IMS examination. Tissue corrosion was avoided by sublimation of MALDI matrix onto the tissue. No solvent was used. The Shimadzu Axima, TOF² was operated in reflectron mode (accelerating voltage 20 kV, reflectron voltage 25 kV). Each spectrum represents the cumulative average of 50 profiles (spectra) per spot with a 70μ spot interval in a rectangle serpentine raster of each tissue section. The calibrated mass accuracy in reflectron mode was ±0.3 Da, or 50 ppm for the parent ions, 75% lower than a recent IMS report. (Groseclose et al., Proteomics 2008; 8:3715-24)

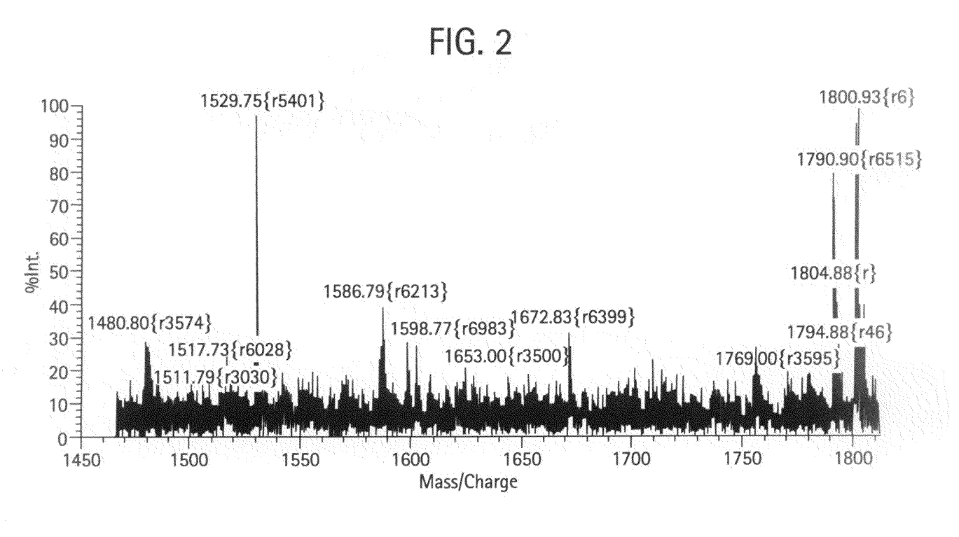

A spectrum obtained from the tumor, and satellite tissue, FIG. 2, was analyzed with Mascot software. The search criteria included only one missed cleavage. Peptide mass fingerprinting, PMF, from the IMS image demonstrated the exact same two proteins, gi|119592539 hCG1787564 [Homo sapiens] Mass: 57590, and gi|119592490 hCG2040674 [Homo sapiens] Mass: 108178 in the colon adenocarcinoma and in the histopathologically normal satellite tissue.

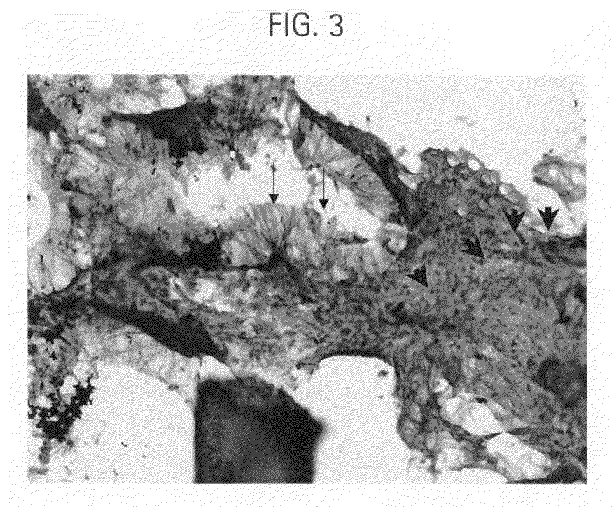

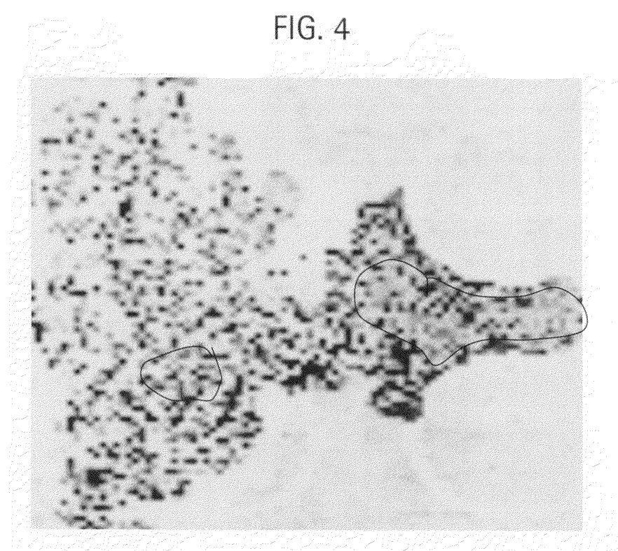

The hematoxylin and eosin stained histological section with the tumor is seen in FIG. 3. The arrowheads outline the edge of the colon adenocarcinoma. The reconstructed IMS image is shown in FIG. 4. The right outlined area corresponds to the tumor in the histology section, and the smaller left outlined region corresponds to the histologically normal satellite tissue. The identical hCG isoforms were identified in both loci. The smaller locus represents a satellite “field defect” that is histologically normal, but biochemically similar to the tumor. A prevailing view of progression from normal tissue to colorectal carcinoma CRC is conversion of abnormal crypt foci with or without intermediate polyps to CRC. We examined several polyps with IMS, and LCMS to identify putative CRC biomarkers.

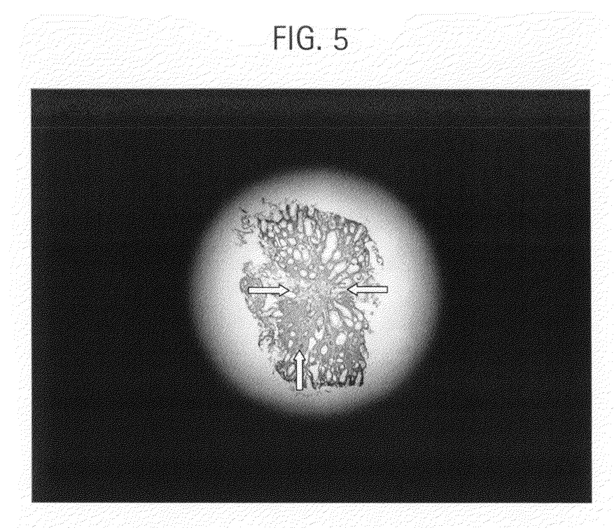

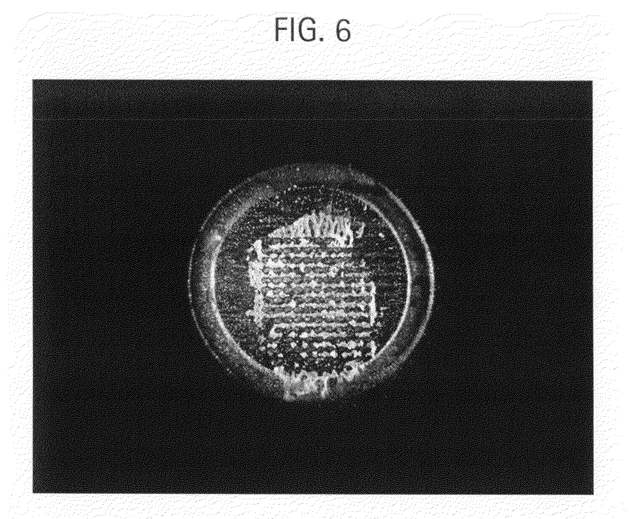

FIG. 5 and FIG. 6 are contiguous sections of colon tissue. The hematoxylin and eosin stained section is demonstrate in FIG. 5, and the sinapic acid matrix sublimated section is demonstrated in FIG. 6. The presence of these two proteins in the histologically normal tissue represents a potential marker for field cancerization or field defect, since these exact same proteins were found in the CRC. The transition from normal colon tissue to CRC is aberrant crypt foci to polyp, to CRC. Therefore, we chose to examine multiple polyps for the presence of putative CRC proteins especially at their periphery, the point of greatest neovascularity and growth. This is the transition zone between the polyp and normal peripheral tissue.

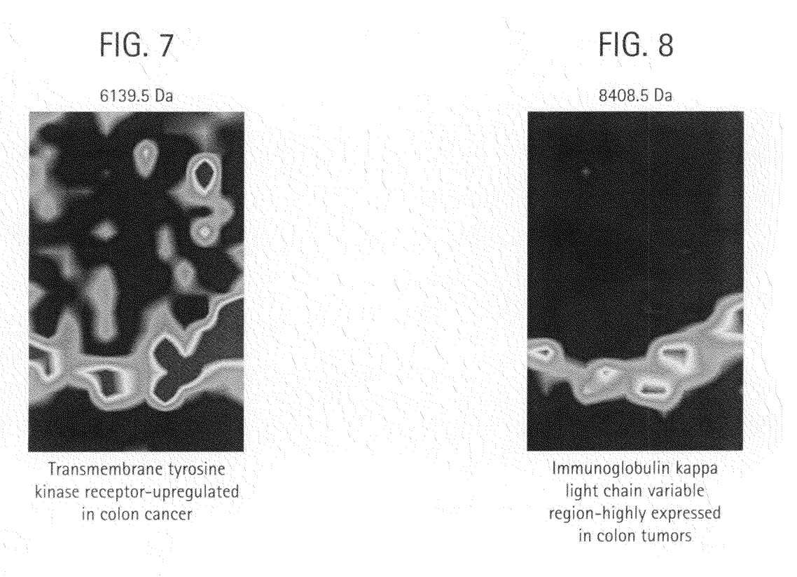

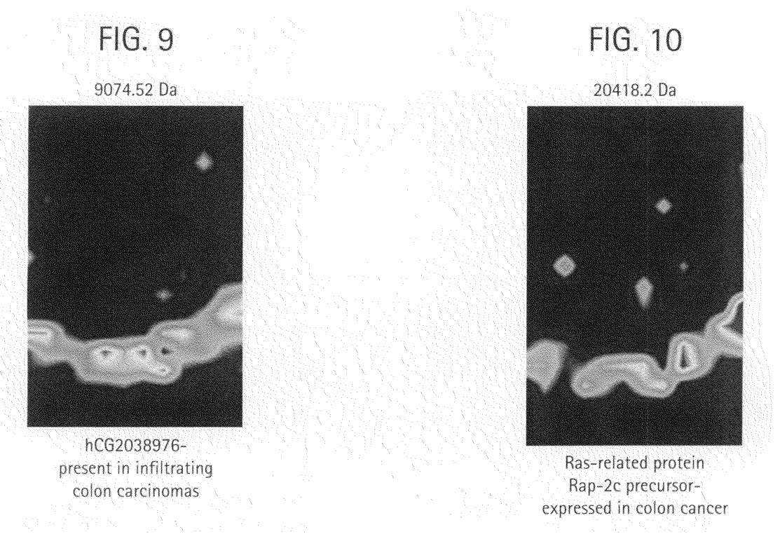

A hematoxylin and eosin stained histologic section of a histopathologically benign appearing colon polyp is shown in FIG. 5. There is a central fibrotic zone and a peripheral zone of increased vascularity at the transition from polyp to normal tissue. IMS of these polyps all revealed tentative protein identifications (obtained by query of the NCBInr database with the mass of the proteins) that were visible at the transition zone between the polyp and the histologically normal peripheral tissue. The proteins are shown in FIGS. 7-10.

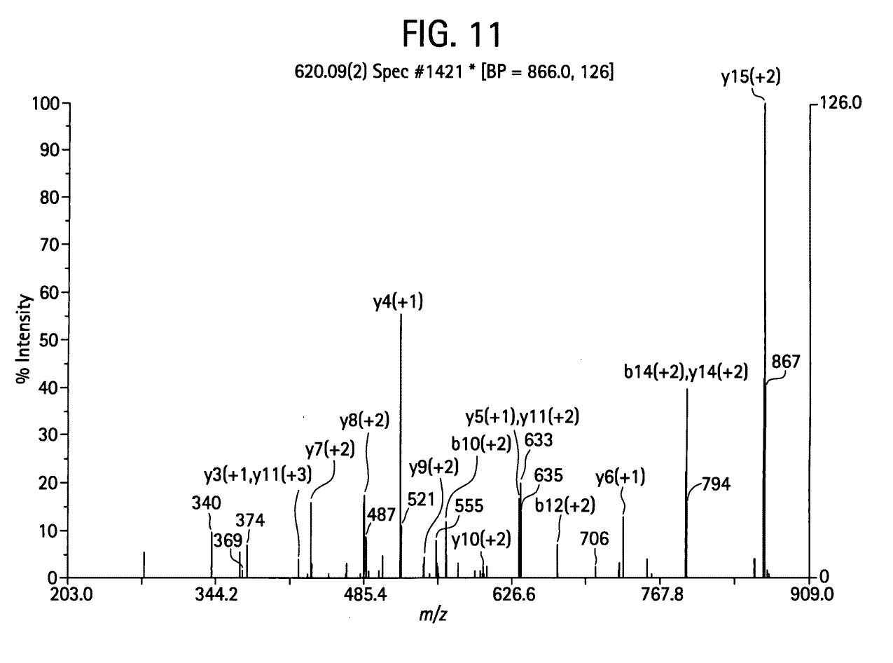

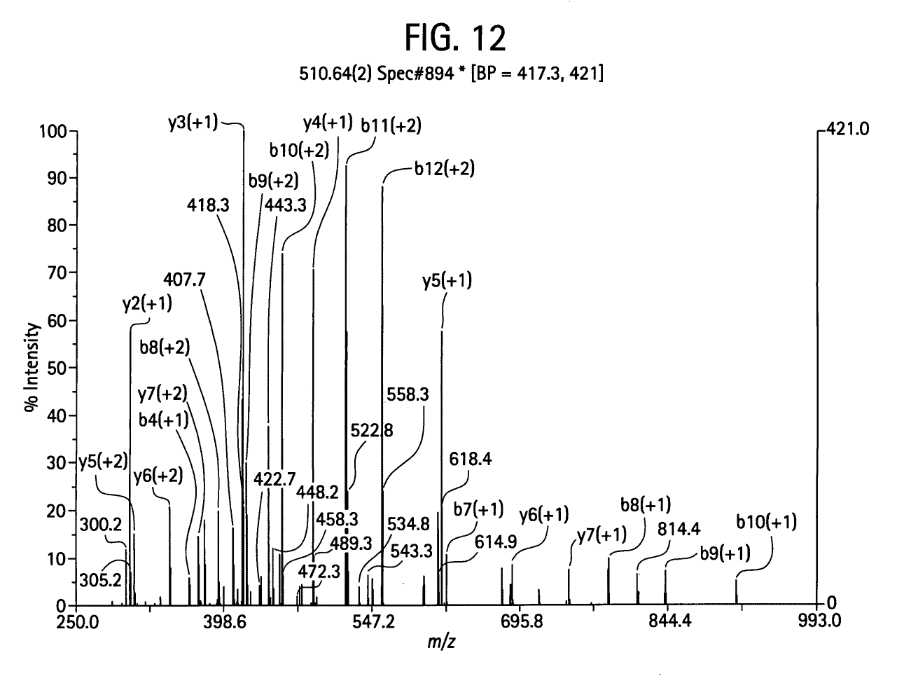

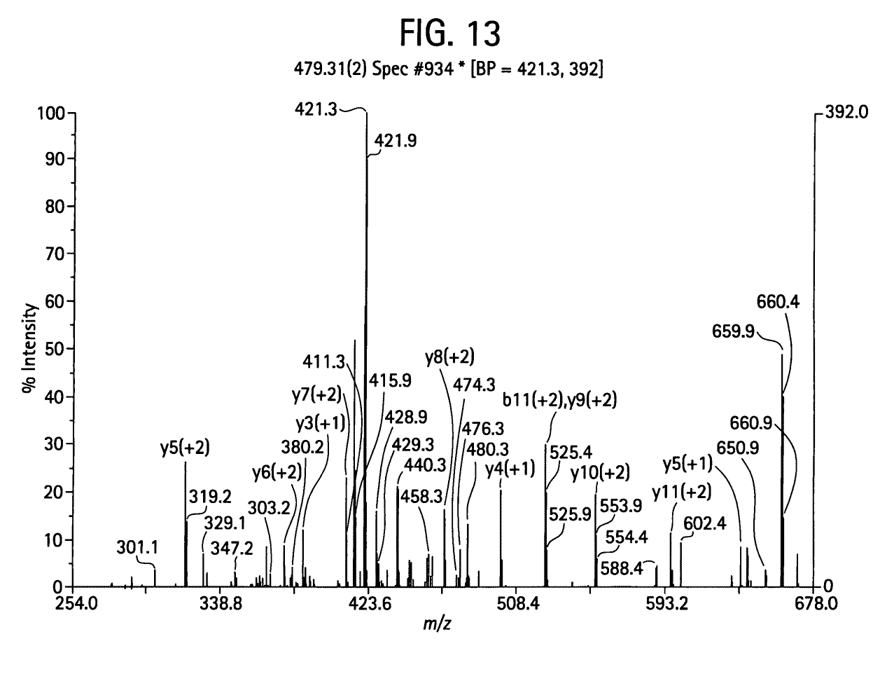

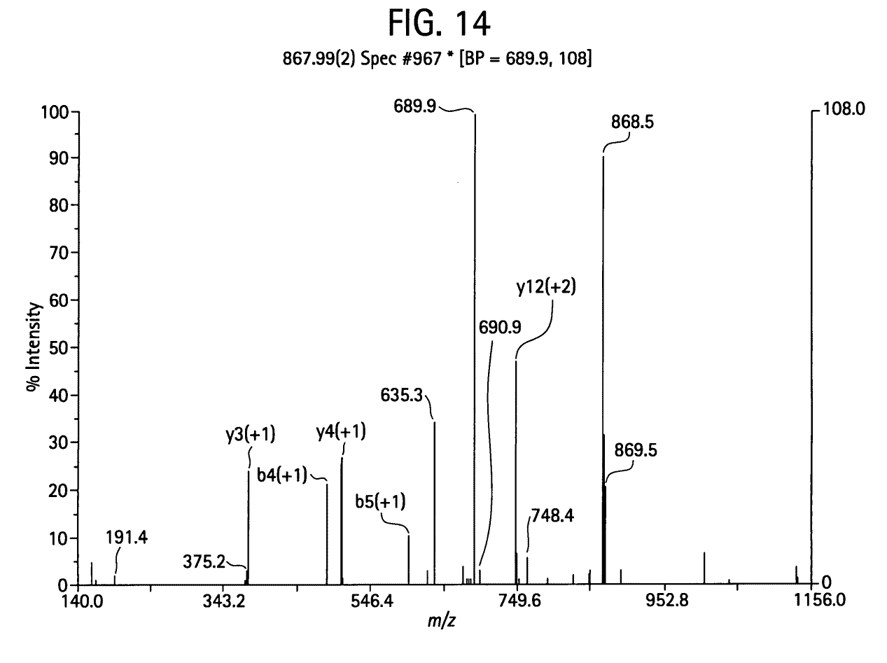

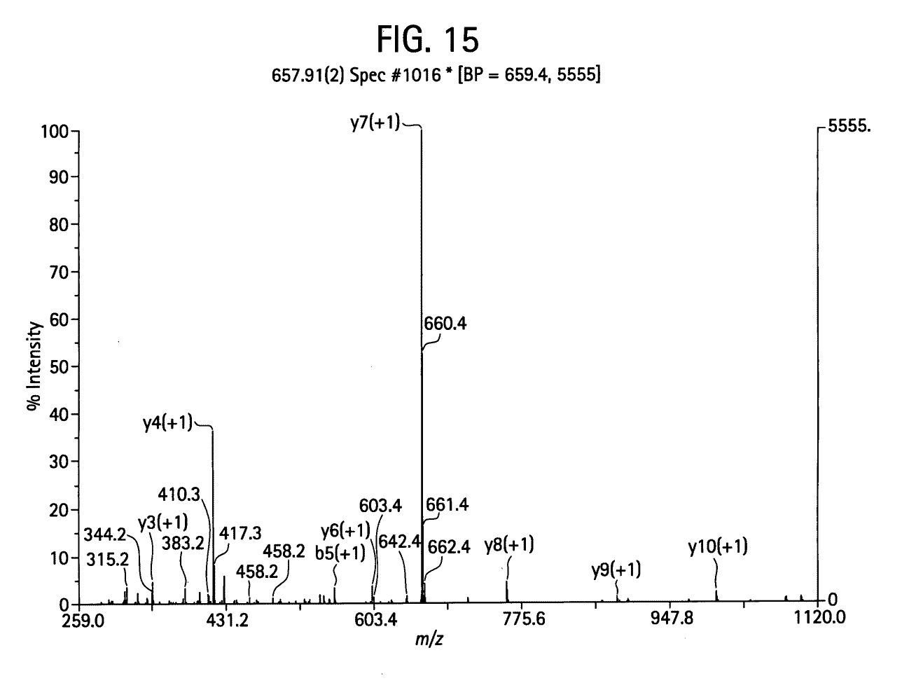

Definitive protein identifications were obtained from trypsin digested tissue protein extracts by LCMS as listed in Tables 1-3. The proteins, marked by an *, are colorectal carcinoma marker proteins. The MSMS spectra of these proteins are shown in FIGS. 11-15.

TABLE 1
Sample A1 Colon Polyp LCMS

GI 30311, cytokeratin 18 (424 AA) [Homo sapiens]
GI 28336, mutant beta-actin (beta'-actin) [Homo sapiens]
*GI 113394, Alcohol deydrogenase beta subunit role in colon cancer
GI 4501881, alpha 1 actin precursor [Homo sapiens]
GI 4757756, annexin A2 isoform 2 [Homo sapiens]
*GI 229751, Chain A, alpha-ferrous-carbonmonoxy, beta-cobaltous-
deoxy hemoglobin (T state).
GI 178027, alpha-actin
GI 63055057, hypothetical protein LOC345651 [Homo sapiens]
GI 6650826, PRO2044 [Homo sapiens]
GI 229752, Chain B, alpha-ferrous-carbonmonoxy, beta-cobaltous-
deoxy hemoglobin (T state)
*GI 40886941, hemoglobin beta [Homo sapiens] colon cancer
proliferation
GI 29446, unnamed protein product [Homo sapiens]

TABLE 2
Sample A2 Colon Polyp LCMS

*GI 229751, chain A, alpha-ferrous-carbonmonoxy, beta-cobaltous-
deoxy hemoglobin (T)
*GI 40886941, hemoglobin beta [Homo sapiens] colon cancer
proliferation
GI 29446, unnamed protein product [Homo sapiens]
GI 58177625, chain B, T-to-T (high) quaternary transitions
in human hemoglobin: betay35f oxy (2 mmIhp, 20% Peg) (1 test set)
GI 61679764, chain B, T-to-T (high) quaternary transitions
in human hemoglobin: betaf42a deoxy low-salt (1 test set)
GI 179409, beta-globin
GI 66473265, beta globin chain [Homo sapiens]
GI 27574235, hemoglobin b, deoxy hemoglobin (A, C:vlm;
B, D:vlm, V67w)
*GI 5453712, galectin 4 [Homo sapiens] (50X lower in colon
cancer than normal)
GI 30908859, actin alpha 1 skeletal muscle protein [Homo
sapiens]
GI 3114508, chain A, R state human hemoglobin [alpha V96w]
GI 61679768, chain B, T-to-T (high) quaternary transitions
in human hemoglobin: betaf45a deoxy low-salt (1 test set)

TABLE 3
Sample A3 Colon Polyp LCMS

GI 4501881, alpha 1 actin precursor [Homo sapiens]
*GI 229751, chain a, alpha-ferrous-carbonmonoxy, k beta-
cobaltous-deoxy hemoglobin (T)
*GI 40886941, hemoglobin beta [Homo sapiens] colon cancer
proliferation
GI 29446, unnamed protein product [Homo sapiens]
*GI 5453712, galectin 4 [Homo sapiens] 50X lower in colorectal
cancer than normal colon
GI 178045, gamma-actin
GI 4501885, beta actin [Homo sapiens]
GI 88953571, PREDICTED: similar to Prostate, ovary, testis
expressed protein on chromosome 2 isoform 2 [Homo sapiens]
GI 113413200, PREDICTED: similar to Prostate, ovary, testis
expressed protein on chromosome 2 [Homo sapiens]
GI 89037243, PREDICTED: similar to actin-like protein [Homo
sapiens]
GI 5901922, cell division cycle 37 proteins [Homo sapiens]
GI 255317, nuclear autoantigen RA33 = A2 hnRNP homolog
[human, Peptide Partial, 25 aa, segment 3 of 4]
GI 106529, Ig kappa chain C region (allotype Inv (1,2))-human (fragment)
GI 32097, unnamed protein product [Homo sapiens]
*GI 37852, vimentin [Homo sapiens] marker for colon cancer

Discussion.

All of these polyps had colorectal carcinoma (CRC) associated marker proteins in the transition zone between polyp and peripheral tissue. This finding indicates that these polyps have already mutated to CRC, and these patients may be at risk for CRC from other patches of aberrant crypt foci that could develop into polyps and CRC. A pathologist reporting no tumor in the normal tissue would be correct. However, histology alone would significantly affect therapy and post operative surveillance by underestimating the extent of metaplastic or malignant disease. IMS and LCMS in conjunction with histopathology will provide a more comprehensive diagnosis for CRC tumors. This diagnostic paradigm provides the patient and his physicians a more complete diagnosis and leads to a better outcome of CRC.

We claim:

1. A method of diagnosing cancer comprising determining the presence of one or more carcinoma associated markers.

2. A method according to claim 1 wherein the carcinoma associated marker is selected from the group consisting of GI 229751, chain A, alpha-ferrous-carbonmonoxy, beta-cobaltous-deoxy hemoglobin (T), GI 40886941, hemoglobin beta [Homo sapiens], GI 5453712, galectin 4 [Homo sapiens] 50×, GI 113394, alcohol deydrogenase beta subunit, GI 40886941, hemoglobin beta [Homo sapiens], GI 5453712, galectin 4 [Homo sapiens] (50× lower in colorectal cancer than normal colon), GI 37852, vimentin [Homo sapiens], GI 119592539 hCG1787564 [Homo sapiens], GI 119592490 hCG2040674 [Homo sapiens], GI 51491284, and GI 112699425 immunoglobulin heavy chain variable region [Homo sapiens].

3. A method according to claim 1 wherein the sample is selected from the group consisting of blood and a tissue biopsy.

4. A method according to claim 1 wherein determining the presence of one or more carcinoma associated markers in a sample is performed by Matrix assisted laser desorption ionization (MALDI) mass spectrometry.

5. A method according to claim 1 wherein determining the presence of one or more carcinoma associated markers in a sample is performed by (a) contacting the sample with an antibody that specifically binds to a carcinoma associated marker permitting formation of a complex between the antibody and the carcinoma associated marker; and (b) measuring the amount of complexes formed, thereby determining the amount of the carcinoma associated marker in the sample.

6. A method according to claim 4 or 5 further comprising comparing the amount of carcinoma associated marker in the sample with either (i) the amount determined for normal samples or (ii) the amount determined for samples obtained from tissue known to contain cancerous cells, wherein the relative abundance of the carcinoma associated marker in the sample indicates that the tissue contains cancer cells.

7. A method of predicting whether a tissue is likely to become cancerous in the future comprising determining the presence of one or more carcinoma associated markers in a sample.

8. A method according to claim 7 wherein the carcinoma associated marker is selected from the group consisting of GI 229751, chain A, alpha-ferrous-carbonmonoxy, beta-cobaltous-deoxy hemoglobin (T), GI 40886941, hemoglobin beta [Homo sapiens], GI 5453712, galectin 4 [Homo sapiens] 50×, GI 113394, alcohol deydrogenase beta subunit, GI 40886941, hemoglobin beta [Homo sapiens], GI 5453712, galectin 4 [Homo sapiens] (50× lower in colorectal cancer than normal colon), GI 37852, vimentin [Homo sapiens], GI 119592539 hCG1787564 [Homo sapiens], GI 119592490 hCG2040674 [Homo sapiens], GI 51491284, and GI 112699425 immunoglobulin heavy chain variable region [Homo sapiens].

9. A method according to claim 7 wherein the sample is selected from the group consisting of blood and a tissue biopsy.

10. A method according to claim 7 wherein determining the presence of one or more carcinoma associated markers in a sample is performed by Matrix assisted laser desorption ionization (MALDI) mass spectrometry.

11. A method according to claim 7 wherein determining the presence of one or more carcinoma associated markers in a sample is performed by (a) contacting the sample with an antibody which specifically binds to a carcinoma associated marker under conditions permitting formation of a complex between the antibody and the molecular carcinoma associated marker; and (b) measuring the amount of complexes formed, thereby determining the amount of the carcinoma associated marker in the sample.

12. A method according to claim 10 or 11 further comprising comparing the amount of carcinoma associated marker in the sample with either (i) the amount determined for normal samples or (ii) the amount determined for samples obtained from a tissue known to contain cancer cells, wherein the relative abundance of the carcinoma associated marker in the sample indicates a greater than normal likelihood that the sample will become cancerous in the future.

13. A method of determining whether a tissue should be surgically resected comprising determining the presence of one or more carcinoma associated markers in a sample.

14. A method according to claim 13 wherein the carcinoma associated marker is selected from the group consisting of GI 229751, chain A, alpha-ferrous-carbonmonoxy, beta-cobaltous-deoxy hemoglobin (T), GI 40886941, hemoglobin beta [Homo sapiens], GI 5453712, galectin 4 [Homo sapiens] 50×, GI 113394, alcohol deydrogenase beta subunit, GI 40886941, hemoglobin beta [Homo sapiens], GI 5453712, galectin 4 [Homo sapiens] (50× lower in colorectal cancer than normal colon), GI 37852, vimentin [Homo sapiens], GI 119592539 hCG1787564 [Homo sapiens], GI 119592490 hCG2040674 [Homo sapiens], GI 51491284, and GI 112699425 immunoglobulin heavy chain variable region [Homo sapiens].

15. A method according to claim 13 wherein the sample is selected from the group consisting of blood and a tissue biopsy.

16. A method according to claim 13 wherein determining the presence of one or more carcinoma associated markers in a sample is performed by Matrix assisted laser desorption ionization (MALDI) mass spectrometry.

17. A method according to claim 13 wherein determining the presence of one or more carcinoma associated markers in a sample is performed by (a) contacting the sample with an antibody which specifically binds to a carcinoma associated marker under conditions permitting formation of a complex between the antibody and the molecular carcinoma associated marker; and (b) measuring the amount of complexes formed, thereby determining the amount of the carcinoma associated marker in the sample.

18. A method according to claim 16 or 17 further comprising comparing the amount of carcinoma associated marker in the sample with either (i) the amount determined for normal samples or (ii) the amount determined for samples obtained from a tissue known to contain cancer cells, wherein the relative abundance of the carcinoma associated marker in the sample indicates a greater than normal likelihood that the sample will become cancerous in the future.

19. A method of determining the likelihood that cells in a biological sample may become cancerous comprising determining the presence of one or more carcinoma associated markers in a sample.

20. A method according to claim 19 wherein the carcinoma associated marker is selected from the group consisting of GI 229751, chain A, alpha-ferrous-carbonmonoxy, beta-cobaltous-deoxy hemoglobin (T), GI 40886941, hemoglobin beta [Homo sapiens], GI 5453712, galectin 4 [Homo sapiens] 50×, GI 113394, alcohol deydrogenase beta subunit, GI 40886941, hemoglobin beta [Homo sapiens], GI 5453712, galectin 4 [Homo sapiens] (50× lower in colorectal cancer than normal colon), GI 37852, vimentin [Homo sapiens], GI 119592539 hCG1787564 [Homo sapiens], GI 119592490 hCG2040674 [Homo sapiens], GI 51491284, and GI 112699425 immunoglobulin heavy chain variable region [Homo sapiens].

21. A method according to claim 19 wherein the sample is selected from the group consisting of blood and a tissue biopsy.

22. A method according to claim 19 wherein determining the presence of one or more carcinoma associated markers in a sample is performed by Matrix assisted laser desorption ionization (MALDI) mass spectrometry.

23. A method according to claim 19 wherein determining the presence of one or more carcinoma associated markers in a sample is performed by (a) contacting the sample with an antibody which specifically binds to a carcinoma associated marker under conditions permitting formation of a complex between the antibody and the molecular carcinoma associated marker; and (b) measuring the amount of complexes formed, thereby determining the amount of the carcinoma associated marker in the sample.

24. A method according to claim 22 or 23 further comprising comparing the amount of carcinoma associated marker in the sample with either (i) the amount determined for normal samples or (ii) the amount determined for samples obtained from a tissue known to contain cancer cells, wherein the relative abundance of the carcinoma associated marker in the sample indicates a greater than normal likelihood that the sample will become cancerous in the future.

25. A diagnostic kit for diagnosing cancer comprising a means for detecting the presence or the quantity of a carcinoma associated marker and instructions correlating the presence or the quantity of the carcinoma associated marker with the likelihood that a sample contains cancerous cells or contains cells that will become cancerous in the future.
